(19) United States
(12) Patent Application Publication
(10) Pub. No.: US 2018/0068088 A1
(43) Pub. Date: Mar. 8, 2018

(54) Vaccination Kiosk

(71) Applicant: Keith Webb, Rockwall, TX (US)

(72) Inventor: Keith Webb, Rockwall, TX (US)

(21) Appl. No.: 15/255,882

(22) Filed: Sep. 2, 2016

(51) Int. Cl. G06F 19/00; A61M 5/30; A61D 7/00; G06Q 20/18; G06Q 20/34

(52) U.S. Cl. CPC G06F 19/3468; A61M 5/30; A61D 7/00; G06F 19/325; G06Q 20/18; G06Q 20/34; A61M 2250/00; A61M 2205/50; A61M 2205/505; A61M 2230/50; A61M 2205/8293; A61M 2205/6054; A61M 2230/005; A61M 2202/30

(57) ABSTRACT

A vaccination kiosk having an interactive touch screen display allows a user to select one or more vaccines, medications, and/or pet care products to be dispensed and administered to a user-identified pet species. The vaccination kiosk further comprises a refrigerated housing or other product housing for storing the vaccines, medications, and/or pet care products, a magnetic card reader or similar payment reader for accepting and verifying payment for the vaccines, a dispensing means for dispensing selected vaccines, medications, and/or pet care products, a jet injector or similar vaccinating means for administering the vaccines, and a powering means to power the vaccination kiosk. In other implementations, a method for dispensing vaccines from a kiosk may comprise receiving profile information from a user relating to a species to be vaccinated, providing the user with a list of vaccines available in the kiosk for the selected species, receiving a request from a user for at least one vaccine based on the list of vaccines available, receiving payment from the user through a magnetic card reader or similar payment system, authorizing the payment, and dispensing one or more vaccines from said kiosk for on-site administration of vaccines to the selected species. In another implementation, a method of dispensing pet care products form a kiosk may comprise receiving profile information from a user relating to a pet species, providing inventory information relating to pet care products available in the kiosk for a selected species, receiving a request from the user for at least one pet care product, receiving payment information through a payment reading system, authorizing the payment information, and dispensing the pet care product from the kiosk.

The present disclosure relates to apparatus and methods for dispensing and administering vaccines and other medications and/or products to a species. More particularly, the invention relates to a kiosk assembly and method related thereto for dispensing and administering species-specific vaccines and dispensing other medications and/or products, such as flea medications, vitamins, and other similar over-the-counter treatments, for household pets.

### BACKGROUND

Conventionally, pets receive medical treatment, including vaccinations and other non-emergency care, at veterinary clinics or other veterinary facilities. In order for such treatment to be obtained, the pet owner must call the facility in advance to schedule an appointment (wherein the scheduled date may or may not be convenient for the owner), often wait in a queue at the clinic or facility with numerous other pet patients that are waiting to be seen, and pay excessive fees, even for the most basic and routine treatments such as vaccinations.

Therefore, there is a need for a more convenient method of obtaining and administering vaccines for pets, without the difficulty associated with scheduling an appointment and waiting in a clinic setting.

### SUMMARY

The present disclosure relates to apparatus and methods for dispensing and administering vaccines and dispensing other medications and/or products for pets through a kiosk.

In some implementations, the apparatus may comprise a vaccination kiosk having an interactive touch screen display which allows a user to select one or more vaccines, medications, and/or pet care products to be dispensed for a user-selected species of pet, a refrigerated housing for storing the vaccines, medications, and/or pet care products that may require refrigeration, a jet injector or similar vaccinating means for administering the vaccines to the species, a magnetic card reader or other payment system for accepting and verifying payment, and a dispensing system for dispensing said at least one vaccine, medication, and/or pet care product to said user.

The vaccination kiosk may further comprise a transmitting means for transmitting a record of the dispensed vaccine, medication, and/or pet care product to the user, a weighing scale to weigh the species to be vaccinated, a thermometer for checking the temperature of the species to be vaccinated, and cameras for visually documenting vaccine administration.

In other implementations, a method for dispensing vaccines from a kiosk may comprise receiving profile information from a user relating to a species of pet to be vaccinated, providing the user with a list of vaccines available in the kiosk for the species, receiving a request from the user for at least one vaccine based on the list of vaccines available in the kiosk, receiving payment from the user through a magnetic card reader or similar payment system, authorizing the payment, dispensing one or more vaccines for the species from the kiosk, and providing means for administering the one or more vaccines to the species.

In another implementation, a method for dispensing pet care products from a kiosk may comprise receiving profile information from a user relating to a pet species, providing inventory information relating to pet care products available in the kiosk for the species, receiving a request from the user for at least one pet care product based on the inventory information, receiving payment from the user through a payment reading system, authorizing payment through a system server coupled to the payment reading system, and dispensing one or more pet care products from the kiosk for the species.

The details of one or more implementations are set forth in the accompanying drawings and the description below. Other features, objects, and advantages of the implementations will be apparent from the description and drawings.

### BRIEF DESCRIPTION OF THE DRAWINGS

For a more complete understanding of this disclosure and its features, reference is now made to the following description, taken in conjunction with the accompanying drawings, in which:

FIG. 1 illustrates a vaccination kiosk system according to the present invention.

FIG. 2 illustrates a front perspective view of a front wall of a vaccination kiosk.

FIG. 3 illustrates a perspective view of the interior of the front wall of the vaccination kiosk.

FIG. 4 illustrates a front perspective view of a back wall of the vaccination kiosk.

FIG. 5 illustrates a side perspective view of the front wall and the back wall of the vaccination kiosk.

### DETAILED DESCRIPTION

Embodiments of the present invention generally provide apparatus and methods for dispensing and administering pet vaccines, and dispensing other pet medications and/or pet care products through a kiosk. The vaccination kiosk may comprise a plurality of different features and components as described herein.

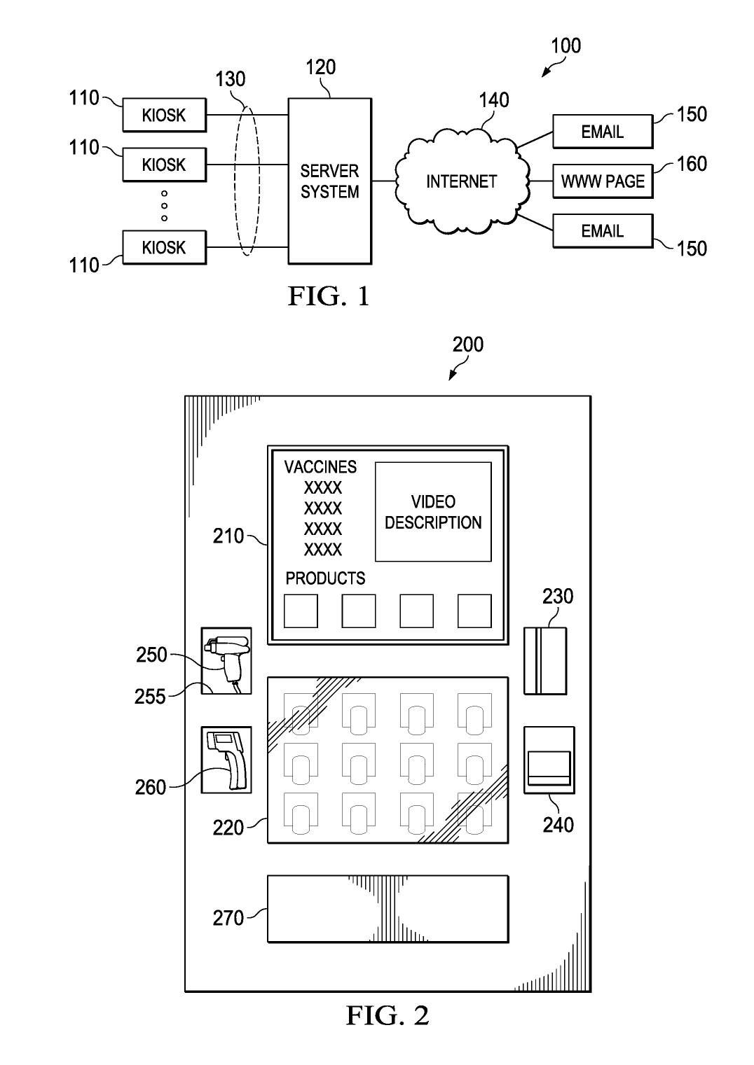

FIG. 1 is a vaccination kiosk system 100 according to a preferred embodiment of the present invention. Vaccination kiosk system 100 may include a plurality of vaccination kiosks 110 coupled to a system server 120 through a virtual network 130. The system server may also be connected to the internet 140 which links the system with individual email accounts 150. The system server may also support a world wide web page 160 for access by users using the internet.

Each vaccination kiosk 110 may further comprise a computer system which enables and manages the information transferred between the user and the vaccination kiosk system. As is known in the art, the computer system may comprise, for example, a central processing unit for processing information, a memory device for storing instructions, a data storage device for storing data, a display device having alphanumeric input capability, a magnetic card reader for reading magnetically imprinted cards, a printing system for printing transaction results and vaccination history of a pet, and a bus system for communicating between system components. Any device suitable for uniquely identifying a customer such as a retinal scanning device, a smart-card reader or finger-print scanner for example, may also be substituted for magnetic card reader.

Turning now to FIG. 2, there is shown a simplified diagram of a front wall view 200 of vaccination kiosk 110 according to one embodiment of the present invention. Front wall 200 of vaccination kiosk 110 may comprise a touch screen display 210, a product housing system 220, a magnetic card reading system 230, a record printing system 240, a jet injection system 250, a thermometric system 260, and a product dispenser slot 270. Each of these components will be described in detail below. Although these components, in conjunction with FIG. 2, are described and shown on the front wall 200 of vaccination kiosk 110, it is noted that each and/or any of these components may be positioned on any wall, in any manner, and in any orientation, inside or outside the kiosk walls in accordance with the spirit of the invention, and the present invention and its components are not limited to any specific positioning, manner, or orientation depicted herein.

Touch screen display 210 provides a user with an interface with which to communicate with the vaccination kiosk 110. For example, touch screen display 210 with alphanumeric input capability may allow the user to create a new pet profile or update an existing pet profile for each pet that user wishes to vaccinate, or for which the user wishes to purchase medications and/or other pet care products, using vaccination kiosk 110. A pet profile may include the user's email address and/or other login information, password, billing information, and pet information such as species, breed, age, color, weight, and pre-existing medical conditions of the pet, e.g., allergies, chronic illness, and other medical information. Thus, certain allergies or medical conditions of a pet that may preclude certain vaccinations or medications in a clinic setting may be highlighted and/or coded by the vaccination kiosk system, so that such vaccines and/or medications are not dispensed or administered from the vaccination kiosk. While touch screen display 210 allows a user to create or manage a pet profile on-site at the vaccination kiosk, it may also be understood that a user may create or update the pet profile online through other means known in the art, such as an internet webpage, a mobile application, etc.

Touch screen display 210 may also communicate to the user by providing information relevant to pet health. For example, after a user logs into an account associated with a particular pet profile, touch screen display 210 may list vaccines, pet medications and/or pet care products available through the vaccination kiosk 120 which would be appropriate for the particular pet, thereby enabling the user to select the product(s) he/she wishes to purchase. Touch screen display 210 may also provide information and/or educational videos for the user relating to general pet care, administration of vaccines, and general information and/or tutorials relating to kiosk use.

Next, front wall 200 of vaccination kiosk 110 may also display product housing system 220 which houses non-vaccine pet products such as vitamins, flea medications, and other over-the-counter medications, which may be purchased and dispensed from the vaccination kiosk. Product housing system 220 may resemble traditional vending machine housing, wherein products may be arranged according to categories, such as species type, medication type, product type, or other similar groupings. Product housing 220 may be refrigerated or non-refrigerated, depending on the thermal requirements of the non-vaccine pet medications and/or pet care products to be housed and dispensed from the vaccination kiosk. After a user selects and pays for a particular pet medication or other non-vaccine pet care product, the product is dispensed through product dispenser slot 270.

Additionally, front wall 200 of vaccination kiosk 110 also includes a magnetic card reading system 230 and record printing system 240. Magnetic card reading system 230 may be configured to transact purchases, including vaccine and non-vaccine products, from the vaccination kiosk by accepting credit cards, debit cards, or other payment cards having a magnetic strip. As is known in the art, magnetic card reader 230 reads and imports data associated with the payment card to a verification system. Upon authorization of the payment card by the verification system, the transaction is approved and dispensation of the purchased products is initiated. Record printing system 240 releases a paper record with information relating to the vaccines, medications, and/or pet care products purchased, total cost, and a past history of vaccines dispensed and administered by the vaccination kiosk for the selected pet. Alternatively, vaccination kiosk 110 may email an electronic record with the same information to the user's email account, as provided in the pet profile. According to another embodiment of the invention, a user may purchase and pre-pay for vaccines, non-vaccine medications, and/or pet care products online using a computer, mobile phone, mobile application, or other means known in the art. Upon arrival at the vaccination kiosk, the user may use the touch screen display to login and initiate dispensing of the pre-paid vaccine and/or non-vaccine products.

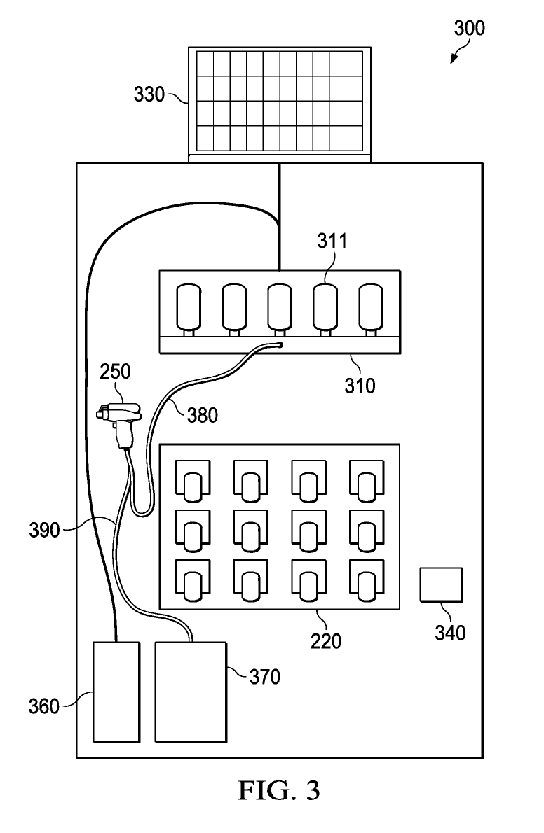

Front wall 200 of vaccination kiosk 110 may also include one or more jet injectors 250, which is a preferred method of administering vaccines through the vaccination kiosk 110. Each jet injector 250 may be stored in a same or separate jet injector compartment 255. As shown in FIG. 3, a jet injector 250 is connectedly attached to the interior of the vaccination kiosk by hoses 380 and 390. Thus, in operation, jet injector 250 may be pulled out of compartment 255 to the length allowed by hoses 380 and 390. After use, hoses 380 and 390 may be threaded back through the compartment and jet injector 250 may be placed back into compartment 255.

As is known in the art, jet injector 250 is a needle-free injection device that uses force generated by compressed gas (including but not limited to carbon dioxide, nitrogen, or a variety of other gases known and used in the art), to propel the vaccine at a high velocity through a small orifice in the skin of the pet. An ultra-fine stream of fluid or powder penetrates the skin and delivers the vaccine in a fraction of a second to the skin, subcutaneous tissue, and underlying shallow muscle. Because jet injectors do not require a needle, they may offer enhanced safety for the user and minimize stress and injection lesions for the pet. Although jet injection offers one form of vaccine administration, there are many other methods of vaccine administration known in the art. Thus, the present disclosure is not intended to be limited to the specific method of jet injection disclosed herein.

Front wall 200 of vaccination kiosk may further include thermometric system 260, which may be used to monitor the body temperature of the pet. If the pet has an elevated temperature indicative of fever, then the thermometric system 260 may issue an alert advising the user to delay administration of the vaccine until the fever has subsided. Alternatively, the alert may be issued and displayed on the touch screen display 210, or by any other means known in the art.

With reference now to FIG. 3, there is shown an internal view 300 of the front wall of the vaccination kiosk 110. Refrigeration unit 310 stores vaccine products 311. The same or separate refrigeration unit may also be provided to store non-vaccine medications and/or pet care products which may require refrigeration. Refrigeration unit 310 may be powered by solar panels 330 or by generator 360. As such, vaccination kiosk may be self-contained. Alternatively, refrigeration unit 310 may also be powered by the electrical system of a store or facility in which vaccination kiosk 110 is located. Refrigeration unit 310 may be connected by hose 380 to at least one jet injector 250. Jet injector 250 may further be connected to compressed gas canister 370 by hose 390. As described, compressed gas canister 370 houses the compressed gas which provides the force necessary to propel a vaccine through jet injector 250 and into the skin of the pet. Compressed gas canister 370 may contain one of a many gases known and used in the art for jet injection of vaccines, including carbon dioxide, nitrogen, air, or other compressed gases. As is known in the art, one or more jet injectors may each be connected by one or more hoses to refrigeration unit 310 and/or to compressed gas canister 370. The present disclosure is not to be limited by the number of jet injectors, hoses, refrigeration units, or compressed gas canisters depicted in the figures or described herein.

With further reference to FIG. 3, as previously described, product housing system 220 may store non-vaccine pet products and medications. Finally, paper dispenser 340 is shown, which is coupled to record printing system 240 of FIG. 2. Paper dispenser 340 may include paper or other printing materials known in the art. Record printing system 240 interacts with paper dispenser 340 to release a paper record with information relating to the vaccine and/or non-vaccine products purchased, total cost, and a past history of vaccines dispensed and administered by the vaccination kiosk for the selected pet. It is further noted that each and any of the components referenced in conjunction with FIG. 3 may be positioned on any wall, in any manner, and in any orientation, inside or outside the kiosk walls, and the present invention and its components are not limited to any specific positioning, manner, or orientation depicted herein.

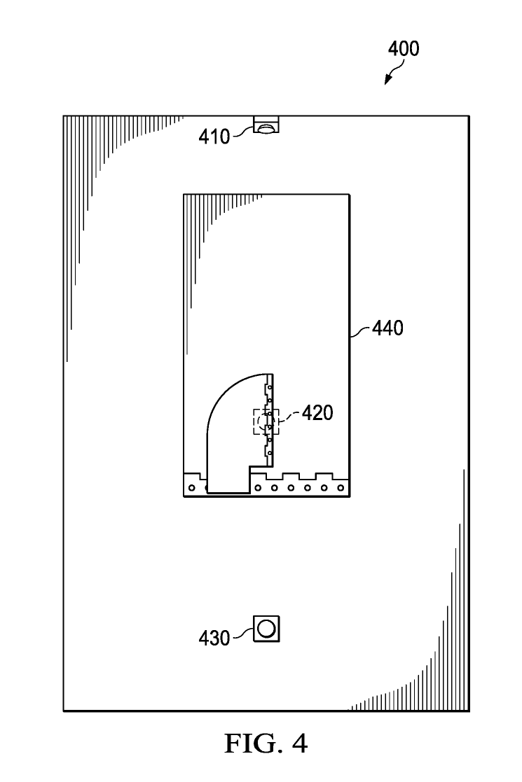

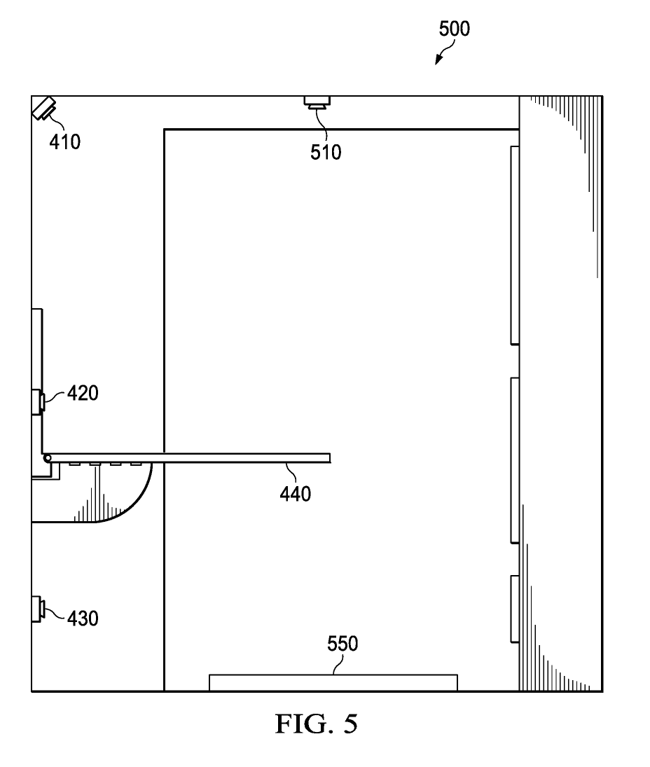

With reference now to FIG. 4, back wall view 400 of vaccination kiosk is shown. Back wall 400 may include security cameras 410, 420, 430 positioned at the top, middle, and bottom of the back wall to provide security and ensure safety within the vaccination kiosk. Cameras 410, 420 and 430 are positioned to record administration of vaccines, thereby visually documenting every use of the vaccination kiosk. Back wall 400 also includes fold down table 440 configured with a weighing scale. Fold down table 440 is mounted to back wall 400 and includes a hinged support which may lock into place when table 440 is pulled down into a horizontal position. Fold down table 440 may be ideal for small pets which satisfy a threshold weight that may be supported by fold down table 440. Securing means may also be provided on the fold-down table 440 to assist in securing the pet. As described in reference to FIG. 5, large pets may be positioned on the floor of the kiosk for vaccine administration.

Referring now to FIG. 5, there is shown an external, side perspective view 400 of the front wall and the back wall of the vaccination kiosk. External view 500 of the vaccination kiosk shows an additional security camera 510 which may be positioned on the ceiling of vaccination kiosk to record administration of vaccines. Directly below camera 510 is weighing scale 550 on the floor of vaccination kiosk. Weighing scale 550 may be ideal for large pets which may exceed the weight limit of fold down table 440. Securing means may be provided on the floor of the kiosk to secure large pets during vaccine administration. As noted above with respect to FIGS. 2 and 3, each of the components referenced in conjunction with FIGS. 4 and 5 may be positioned on any wall, in any manner, and in any orientation, inside or outside the kiosk walls, and the present invention and its components are not limited by any orientation, whether or not depicted herein.

With further reference to FIGS. 1-5, a preferred method of the vaccination kiosk will now be described. As previously described, the touch screen display 210 of vaccination kiosk 110 provides a user-friendly interface wherein consumers may select from a list of product options that are available for purchase. A first-time user may begin by using the touch screen display 210 to create a pet profile for a pet. A pet profile may include (but is not limited to), for example, a username and password, along with general information about the pet, including but not limited to species, breed, age, color, weight, allergies, and other medical history. All profile information entered by the user may be stored in the server system accessible through the virtual network. The pet profile may also be created remotely via mobile app, or via internet using a mobile device, a desktop, or other means known in the art.

Once a pet profile is completed, a new user (or a returning user with an existing pet profile) may log into the vaccination system and upload the associated pet profile. The touch screen display 210 may display a menu of vaccines, medications, and/or other pet care products that may be purchased, specific to the pet associated with the pet profile. Menu options may include categories of vaccines, or categories of pet medications and/or pet care products. Based on the pet profile, the menu may exclude products from the listing that are not appropriate for the given pet. For example, if a pet profile indicates a medication allergy, such medications may not be listed on the touch screen display 210 and thus, may not be dispensed from the kiosk.

Once a user selects a category of interest from the displayed menu, the touch screen display 210 may display sub-categories within the category of interest. The user may use the touch screen display 210 to narrow down a product selection based the specific species, breed, age, and/or vaccine, medication, and/or product needs of the pet. For example, if the user selects a category entitled “Vaccinations”, the touch screen display 210 may list all vaccines available in the vaccination kiosk that may typically be due for the specific age and species of pet. The user is then free to select one or more vaccines to administer to the pet. Alternatively, if the user selects a category entitled, for example, “Pet Medication”, the touch screen display 210 may display all of the types of medications available within the kiosk that may be appropriate for the particular pet. Categories of pet medications and/or pet care products may include, but is not limited to, flea medicines, vitamins, and other over-the-counter pet medicines.

After making one or more product selections, the user may complete the transaction by swiping a debit card, credit card, or other payment card in the magnetic card reader system 230. As is known in the art, the magnetic card reader system 230 verifies payment and authorizes the transaction. If the payment card is approved, the transaction may be processed on the system server, and a dispensing system is activated. If payment is not authorized, the touch screen display 210 may display an error message and the user may attempt to use another form of payment or end the transaction. As described above, a user may also make payment online prior to arriving at the vaccination kiosk. Alternatively, a user may set-up an automatic online payment option, wherein a payment card is charged or debited automatically based on billing information provided in the user's pet profile.

Once the transaction is authorized, the vaccination kiosk may dispense the purchased vaccines, medications and/or pet care products from the vaccination kiosk 110 to the appropriate location. If the products purchased are non-vaccine medications or pet care products, such products may be dispensed from product housing system 220 or refrigeration unit 310 (if the items require refrigeration) through product dispenser slot 270. If the products purchased are vaccines, the selected vaccines may be dispensed from refrigeration unit 310, through hose 380, to jet injector 250, through any means and in any manner known in the art.

For vaccinations, the user may position a pet in an appropriate location for administration of the vaccine. A smaller pet may be placed on fold-down table 440, and held in place for vaccine administration. A larger pet may be placed on the floor scale 550. Securing means may be provided on the fold-down table 440 or near floor scale 550 to secure a moving pet, if necessary. The user may remove jet injector 250 from its compartment 255 and pull to a comfortable position near the pet. The user may administer a first vaccine by simply placing the nozzle of jet injector 250 against a pet skin and pulling a trigger. If multiple vaccine products were purchased by the user, the vaccination kiosk may dispense each vaccine separately, so that multiple vaccines may be administered successively through a single jet injector 250. Alternatively, according to another implementation, the vaccination kiosk may provide multiple jet injectors, one for each type of vaccine to be dispensed. Thus, if multiple vaccines were purchased, the user may successively administer each vaccine from each corresponding jet injector. At any time during this process, user may access the touch screen 210 to view video resources or obtain instruction on administering the vaccine. Moreover, it is to be noted that administration of vaccine(s) through jet injection is known in the art, and the present disclosure does not limit vaccine administration to a pet to any particular method or process of jet injection.

When all of the vaccines purchased by the user have been administered, and/or all of the non-vaccine medications and/or pet care products purchased by the user have been dispensed, touch screen display 210 may indicate that the session has been completed. Upon touch confirmation by the user on the touch screen display 210, the record printing system 240 prints a record of the transaction. The record may include the costs associated with the transaction, a listing of the vaccines, medications, and/or pet care products dispensed, and a history of vaccines previously dispensed and administered though the vaccination kiosk system. The record may also be transmitted via electronic mail to the account provided in the user's pet profile.

It is to be understood that the implementations are not limited to particular systems or processes described which may, of course, vary. For example, the vaccination kiosk need not be limited for use in conjunction with household pets. A variety of animals, species, and even humans may derive benefit from the disclosure set forth herein. Moreover, the invention defined by the above paragraphs is not to be limited to particular details set forth in the above description, as many apparent variations thereof are possible without departing from the spirit or scope of the present invention. It is also to be understood that the terminology used herein is for the purpose of describing particular implementations only and is not intended to be limiting.

Although the present invention has been described in detail, it should be understood that various changes, substitutions, and alterations may be made herein without departing from the spirit and scope of the disclosure as defined by the appended claims. Moreover, the scope of the present application is not intended to be limited to the particular embodiments of the process, machine, manufacture, composition of matter, means, methods, and steps described in the specification. As one of ordinary skill in the art will readily appreciate from the disclosure, the processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method for dispensing vaccines from a kiosk, the method comprising:
receiving, from a user, profile information relating to a species to be vaccinated;
providing inventory information relating to vaccines available in said kiosk for said species;
receiving, from said user, a request for at least one vaccine based on said inventory information;
receiving, from said user, payment information through a payment reading system;
authorizing said payment information though a system server coupled to said payment reading system;
dispensing said at least one vaccine from said kiosk for administration to said species; and
providing means for administering said at least one vaccine to said species at said kiosk.

2. The method of claim 1, wherein said means for administering comprises:
a jet injector system coupled to said kiosk to vaccinate said species.

3. The method of claim 2, further comprising the step of:
powering said jet injector system through a compressed gas canister.

4. The method of claim 1, further comprising the step of:
storing, in said system server, a record of said at least one vaccine dispensed from said kiosk.

5. The method of claim 1, further comprising the step of:
transmitting, to said user, a record of said at least one vaccine dispensed from said kiosk.

6. The method of claim 5, wherein said step of transmitting comprises:
emailing said record to said user.

7. The method of claim 5, wherein said step of transmitting comprises:
printing a paper copy of said record from said vaccination kiosk.

8. The method of claim 1, wherein the step of receiving profile information further comprises:
receiving information relating to age and species type of said species.

9. The method of claim 1, wherein the step of receiving profile information further comprises:
receiving information relating to medical history of said species.

10. The method of claim 1, wherein said payment reading system comprises:
a magnetic card reader.

11. The method of claim 1, further comprising the step of:
prior to dispensing said at least one vaccine, approving a request for at least one vaccine based on profile information relating to the species to be vaccinated.

12. The method of claim 1, further comprising the steps of:
testing a temperature of said species to be vaccinated; and
prior to dispensing said at least one vaccine, approving a request for at least one vaccine based on said temperature of said species to be vaccinated.

13. The method of claim 1, further comprising the step of:
visually documenting use of said kiosk using a camera system.

14. An automated vaccination kiosk for dispensing and administering a plurality of vaccines, comprising:
a vaccination housing having a refrigeration unit for storing said plurality of vaccines;
an interactive display for communication with a user, wherein said interactive display allows said user to select at least one vaccine from said plurality of vaccines to be dispensed for a selected species;
a payment system for accepting and verifying payment for said at least one vaccine;
a dispensing system for dispensing said at least one vaccine to said user;
a vaccinating means coupled to said dispensing system for administering said at least one vaccine to said selected species; and
a powering means to power said vaccination kiosk.

15. The automated vaccination kiosk of claim 14, wherein said interactive display comprises:
a touch screen tablet.

16. The automated vaccination kiosk of claim 14, wherein said vaccinating means comprises:
a jet injector system.

17. The automated vaccination kiosk of claim 16, wherein said jet injector is powered by a compressed gas canister.

18. The automated vaccination kiosk of claim 14, wherein said payment system comprises:
a magnetic card reader.

19. The automated vaccination kiosk of claim 14, wherein said powering means comprises:
a solar panel.

20. The automated vaccination kiosk of claim 14, wherein said powering means comprises
a generator.

21. The automated vaccination kiosk of claim 14, further comprising:
a weighing scale to weigh said selected species.

22. The automated vaccination kiosk of claim 21, wherein said weighing scale is mounted on a fold-down table.

23. The automated vaccination kiosk of claim 14, further comprising:
a thermometric reader for testing the body temperature of said selected species.

24. The automated vaccination kiosk of claim 14, further comprising:
a transmitting means to transmit a record of said at least one vaccine dispensed by said vaccination kiosk to said user.

25. The automated vaccination kiosk of claim 24, wherein said transmitting means comprises:
a printer.

26. The automated vaccination kiosk of claim 24, wherein said transmitting means comprises:
an electronic mail.

27. The automated vaccination kiosk of claim 14, further comprising:
at least one camera for visually documenting use of said vaccination kiosk.

28. A method for dispensing pet care products from a kiosk, the method comprising:
receiving, from a user, profile information relating to a pet species;
providing inventory information relating to pet care products available in said kiosk for said species;
receiving, from said user, a request for at least one pet care product based on said inventory information;
receiving, from said user, payment information through a payment reading system;
authorizing said payment information though a system server coupled to said payment reading system; and
dispensing said at least one pet care product from said kiosk for said species.

29. The method of claim 28, further comprising the step of:
storing, in said system server, a record of said at least one pet care product dispensed from said kiosk.

30. The method of claim 28, further comprising the step of:
transmitting, to said user, a record of said at least one pet care product dispensed from said kiosk.

31. The method of claim 30, wherein said step of transmitting comprises:
emailing said record to said user.

32. The method of claim 30, wherein said step of transmitting comprises:
printing a paper copy of said record from said vaccination kiosk.

33. The method of claim 28, wherein the step of receiving profile information further comprises:
receiving information relating to age and species type of said species.

34. The method of claim 28, wherein the step of receiving profile information further comprises:
receiving information relating to medical history of said species.

35. The method of claim 28, wherein said payment reading system comprises:
a magnetic card reader.
